(19) United States
(12) Patent Application Publication
(10) Pub. No.: US 2026/0224614 A1
(43) Pub. Date: Aug. 6, 2026

(54) REACTED MATRIX FOR CELL DISPERSION

(71) Applicant: THE TEXAS A&M UNIVERSITY SYSTEM, College Station, TX (US)

(72) Inventors: Peter P. Nghiem, College Station, TX (US); Aaron B. Morton, College Station, TX (US); Jacob A. Kendra, College Station, TX (US)

(73) Assignee: THE TEXAS A&M UNIVERSITY SYSTEM, College Station, TX (US)

(21) Appl. No.: 19/532,933

(22) Filed: Feb. 6, 2026

Related U.S. Application Data
(60) Provisional application No. 63/754,943, filed on Feb. 6, 2025.

(51) Int. Cl. A61K 33/08; A61K 9/00; A61K 35/13; A61K 35/34; A61K 47/36; A61P 21/00

(52) U.S. Cl. CPC A61K 33/08; A61K 9/0019; A61K 35/13; A61K 35/34; A61K 47/36; A61P 21/00

(57) ABSTRACT

A method of making a composition; a composition made according to the methods; a composition comprising cells; and a bioactive glass composition comprising an amorphous calcium polyphosphate phase; a suspension comprising a liquid; and a bioactive glass composition; and a method of treating injured or diseased tissue are described. In examples, the method of making comprises combining cells and a bioactive glass composition comprising an amorphous calcium polyphosphate phase. In an example, the method comprises preparing a mixture comprising about 10 wt. % to about 40 wt. % B₂O₃; about 15 wt. % to about 40 wt. % P₂O₅; about 10 wt. % to about 25 wt. % CaO; and about 5 wt. % to about 20 wt. % Na₂O; and melting the mixture at a temperature and for a time sufficient to provide the bioactive glass composition.

### CROSS-REFERENCE TO RELATED APPLICATION

This application claims the benefit and priority of U.S. Provisional Application No. 63/754943, filed Feb. 6, 2025, the entire disclosure of which is hereby incorporated by reference in its entirety.

### BACKGROUND

Duchenne Muscular Dystrophy (DMD) is a progressive, muscle-wasting disease, manifesting symptoms in children around 2-3 years of age, in particular, difficulty climbing stairs, a waddling gate, and frequent falls. Disease progression causes most patients to be wheelchair dependent around 10-12 years of age and in need of assisted ventilation by ~age 30.

DMD promotes rapid development of muscle weakness that results in a loss of functional muscle in children. Concomitant with a loss in function is an exhausted myogenic stem cell population. To date, there is no cure for patients with DMD, a situation that is compounded by significant side effects in FDA-approved treatments.

Stem cell therapy is a promising treatment for many diseases, as it has the potential to, in part, replace both the exhausted stem cell pool and future mature cells that stem cells will become through differentiation in response to appropriate cell cycle cues. Severe genetic diseases such as DMD stand to benefit from stem cell replacement. Indeed, current genetic approaches aim to stabilize the patient's current mature muscle cells, but are not capable of growing new, healthy cells. Hence, this is a key reason many milestones are not met in clinical trials. Moreover, AAV gene therapies can be wrought with significant side effects like once in a lifetime treatment, significant immune response, and high cost.

In genetic muscle diseases like DMD as well as many other tissues and corresponding conditions, stem cell treatment has the potential to replace tissue that has been degraded or degenerated. However, several significant challenges remain before widespread and successful stem cell therapy can be implemented. Examples of such challenges include, for example, 1) pocketing of the injected stem cells in tissues of interest, resulting in acute stem cell death, 2) host immune response to injected stem cells, and 3) need for a niche in the target tissue for stem cell implantation. Importantly, ancillary therapies that prevent pocketing and provide a suitable matrix for the stem cell niche are drastically needed.

### SUMMARY

To address these and related challenges, in various aspects, the present disclosure provides a method of making a composition; a composition made according to the methods; a composition comprising cells, and a bioactive glass composition comprising an amorphous calcium polyphosphate phase; a suspension comprising a liquid; and the composition; and a method of treating injured or diseased tissue. In an embodiment, the tissue comprises muscle. In an embodiment, the tissue comprises articular joint cartilage.

In an aspect, the present disclosure provides a method of making a composition. In an embodiment, the method comprises combining cells and a bioactive glass composition comprising an amorphous calcium polyphosphate phase.

In an embodiment, the method further comprises preparing a mixture comprising about 10 wt. % to about 40 wt. % B₂O 3; about 15 wt. % to about 40 wt. % P₂O₅; about 10 wt. % to about 25 wt. % CaO; and about 5 wt. % to about 20 wt. % Na₂O; and melting the mixture at a temperature and for a time sufficient to provide the bioactive glass composition.

In an embodiment, the mixture further comprises about 2 wt. % to about 10 wt. % CoO, about 0.5 wt. % to about 2 wt. % ZnO, about 0.1 wt. % to about 1 wt. % CuO, or a combination thereof.

In an embodiment, the method further comprises calcining the mixture to evolve water prior to the melting.

In an embodiment, the mixture further comprises phosphoric acid.

In an embodiment, melting the mixture is at a temperature in a range of about 900° C. to about 1150° C.

In an embodiment, the method further comprises grinding the bioactive glass composition to provide particles of the bioactive glass composition.

In an embodiment, the particles comprise a largest size of less than about 10 micrometers.

In an embodiment, combining the cells and the bioactive glass composition comprises suspending the particles of the bioactive glass composition and the cells in a liquid.

In an embodiment, the liquid is a sterile saline solution.

In an embodiment, the liquid comprises sodium alginate.

In an aspect, the present disclosure provides a composition made according to the methods of any embodiment of the present disclosure.

In another aspect, the present disclosure provides a composition comprising cells; and a bioactive glass composition comprising an amorphous calcium polyphosphate phase.

In an embodiment, the bioactive glass composition comprises particles of the bioactive glass composition, wherein the particles comprise a largest size of less than about 10 micrometers.

In an embodiment, the cells are or comprise stem cells and the stem cells comprise immortalized stem cells.

In an embodiment, the stem cells comprise myoblasts.

In an embodiment, the stem cells comprise myogenic stem cells.

In an embodiment, the stem cells comprise dystrophin-producing stem cells.

In an embodiment, the cells comprise cancer cells, such as colon cancer cells or breast cancer cells.

In an embodiment, the amorphous calcium polyphosphate phase comprises orthophosphate and pyrophosphate anions.

In an embodiment, the bioactive glass composition is made according to the methods of any embodiments of the present disclosure.

In another aspect, the present disclosure provides a suspension comprising a liquid; and a composition according to any embodiment of the present disclosure.

In an embodiment, the liquid is a sterile saline solution.

In an embodiment, the liquid further comprising sodium alginate.

In yet another aspect, the present disclosure provides a method of treating injured or diseased tissue, the method comprising contacting the injured or diseased tissue with a composition of any embodiment of the present disclosure or a suspension of any embodiment of the present disclosure. In an embodiment, the tissue comprises muscle. In an embodiment, the tissue comprises articular joint cartilage.

In an embodiment, the composition prevents or limits pocketing, and promotes dispersion in injected muscle relative to injection of the cells without the bioactive glass composition.

In an embodiment, any pockets in the muscle comprise a thickness of less than 80 micrometers 1 day to 1 month after injection.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of the subject matter of the present application will become more readily appreciated as the same become better understood by reference to the following detailed description, when taken in conjunction with the accompanying drawings, wherein:

FIG. 1A is a representative image of reacted matrix (RM) in suspension, according to embodiments of the present disclosure, with a scale bar of 10 μm;

FIG. 1B illustrates weight release of RM particles (300 mg) in 50 mL of SBF across 7 days (168 h), according to embodiments of the present disclosure;

FIG. 2A is a diagram of particle degradation, ion release, and RM formation, according to embodiments of the present disclosure, where (left) a borophosphate particle reacts in PSS by releasing ionic species, (middle) released ions react with SBF to form a biocompatible RM of calcium phosphate on the particle surface, and (right) the particle is fully reacted to form a hollow precipitate comprising RM and pyrophosphate anions; the PA in the RM inhibit the formation of hydroxyapatite leaving the biocompatible RM;

FIG. 2B schematically illustrates RM particles binding to adhesion proteins upon cells preventing them from sticking to each other and promoting dispersion and implantation within soft tissue compartments, according to embodiments of the present disclosure;

FIG. 3 is an image of RM particles bound to MyoK9 stem cells, according to embodiments of the present disclosure, where the box marks areas of increased magnification;

FIGS. 4A and 4C are images of stem cells alone in SCID mice showing clumping of stem cells;

FIGS. 4B and 4D are images of stem cells and RM distributed in SCID mice, according to embodiments of the present disclosure;

FIG. 5A is an image of a mass of ischemic induced mesenchymal stem cell (iMSC) cells identified with arrows in healthy muscle;

FIG. 5B is an image of iMSCs distributed in healthy muscle following combination with RM, according to embodiments of the present disclosure;

FIG. 5C is an image of a mass of ischemic iMSC cells identified with arrows in injured muscle;

FIG. 5D is an image iMSCs distributed in injured muscle following combination with RM, according to embodiments of the present disclosure;

FIGS. 6A and 6B are images of biopsy muscle 1 month post stem cell only injection;

FIGS. 6C and 6D are images of a biopsy 1 month post SCRM injection, according to embodiments of the present disclosure, with a scale bar=100 μm;

FIG. 7 illustrates pocketing and acute death of intramuscularly injected human induced pluripotential stem cells (hiPSCs) in the canine Duchenne muscular dystrophy model in the absence of RM, where the affected dog was immunosuppressed and injected with hiPSCs and biopsied one week later. Scale bar=1000 μm;

FIG. 8 is an image of RM adhered to iMSC, according to embodiments of the present disclosure, where the image taken at 20× (N.A. 0.75) on a Leica Confocal microscope. Scale bar 10 μm;

FIGS. 9A-9K: RM leads to viable and dispersed stem cells in porcine articular joints. (9A-9C) Masson's Trichrome staining. (9A) Undamaged, normal joint (arrows indicate some healthy chondrocytes). (9B and 9C) Joints were damaged with BaCl2 and collagenase type I+II. 72 hours later, ~7 MIL iMSCs were injected (with or without RM). (9B) iMSCs injected alone. Dotted outline indicates clumping of injected stem cells. (8C) iMSCs +RM. Dotted outline indicates stem cell dispersal. (9D) Injected intra-articular into the distal interphalangeal joint (arrows around hypodermic needle inserted into joint). Total injection volumes were 0.75-1 mL. (9E-9K) Immunostaining for human mitochondrial antibody to detect iMSCs (9E) Secondary IgG antibody only (control). F-H) iMSC+RM injection led to super-dispersion of viable stem cells that localized with nuclei. (9I-9K) Only iMSCs were injected (no RM) which showed a less amount of viable stem cells, many that did not localize with nuclei. N=2/group;

FIG. 10A is an image of colon cancer cells combined with RM according to embodiments of the present disclosure; and

FIG. 10B is an image of breast cancer cells combined with RM according to embodiments of the present disclosure.

### DETAILED DESCRIPTION

In various aspects, the present disclosure provides a method of making a composition; a composition made according to the methods; a composition comprising cells, and a bioactive glass composition comprising an amorphous calcium polyphosphate phase; a suspension comprising a liquid; and the composition; and a method of treating injured or diseased tissue.

Second generation biomaterials are biologically reactive when placed within a physiological environment. In contrast to first generation (biologically inert and non-reactive), second generation (biologically inert and biologically reactive) biomaterials do not promote gene transcription within the tissue microenvironment. Ion matrices have been used as second generation biomaterials, promoting cellular adhesion for orthopedic and dental applications. In that regard, early cellular adhesion experiments investigated ion matrices as coatings to enhance osteogenic adhesion of implants, discovering that fibronectin residing upon cell membranes forms the primary interaction with reacted matrices (RM). Initial experiments focused upon RM reactions in the context of adhesion to bone.

As demonstrated further herein, it has been surprisingly found that combining RM with cells according to embodiments of the present disclosure assists with distribution of cells in tissue into which such compositions are disposed. Further such compositions avoid or limit pocketing or other aggregations of cells in tissue, such as to increase the survival of the cells implanted in the tissue.

As described further herein, in embodiments, the RM comprises a bioactive glass, such as a bioactive glass comprising a borate phosphate based amorphous non-crystalline solid.

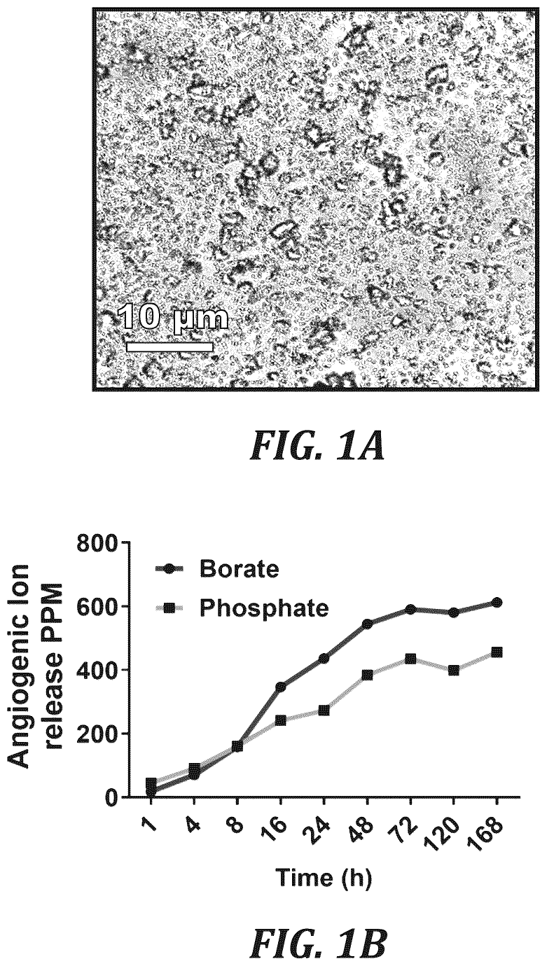

Upon introduction to physiological environments, borophosphate ion matrix particles form a highly biocompatible RM surface product (FIGS. 1A and 1B). Upon complete reaction (total ion release and material conversion) RM surface chemistry becomes highly compatible with glycoproteins like fibronectin. Stem cells designed for implantation and development within diseased or injured soft tissue microenvironments often adhere to each other, promoting ischemic pockets of cells that die rather than disperse for incorporation. As fibronectin is plentiful upon stem cell populations, RM is well suited for stem cell adhesion, as demonstrated herein.

As shown herein, biocompatible RM particles combine with stem cells, such as myogenic stem cells, prevent or limit pocketing, and promote dispersion in injected muscles. Herein the present disclosure demonstrates, in certain embodiments, RM particles combined with stem cells, such as induced mesenchymal stem cells (iMSCs) and myoK9 cells (an immortalized, dystrophin producing, canine myoblast cell line), suspended in sterile saline prior to injection into myofascial compartments promoted healthy stem cell dispersion in tissue and were fully incorporated as mature cells in vivo for up to 1 month.

Mesenchymal stem cells can differentiate into 1) myogenic stem cells (muscle), 2) chrondrocytes (for cartilage), 3) adipocytes (fat), and 4) osteocytes (bone). In an embodiment, the stem cells comprise any one or more of these types of cells.

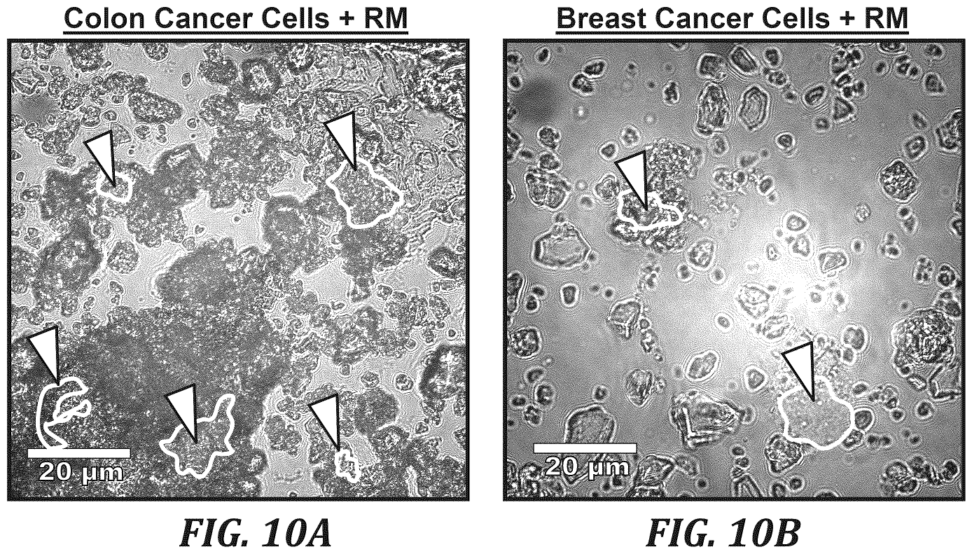

While stem cells combined with RM are discussed herein, it will be understood that other types of cells can be combined with RM, according to embodiments of the present disclosure, and are within the scope of the present disclosure. See, for example, Example 3 and FIGS. 10A and 10B.

In embodiments, the methods of making the bioactive glass compositions and the bioactive glass compositions are described in PCT application no. PCT/US2022/042374, the contents of which are incorporated herein by reference.

In an aspect, the present disclosure provides a method of making a composition. In an embodiment, the method comprises combining cells and a bioactive glass composition comprising an amorphous calcium polyphosphate phase.

In an embodiment, the method further comprises preparing a mixture comprising about 10 wt. % to about 40 wt. % B₂O₃; about 15 wt. % to about 40 wt. % P₂O₅; about 10 wt. % to about 25 wt. % CaO; and about 5 wt. % to about 20 wt. % Na₂O; and melting the mixture at a temperature and for a time sufficient to provide the bioactive glass composition.

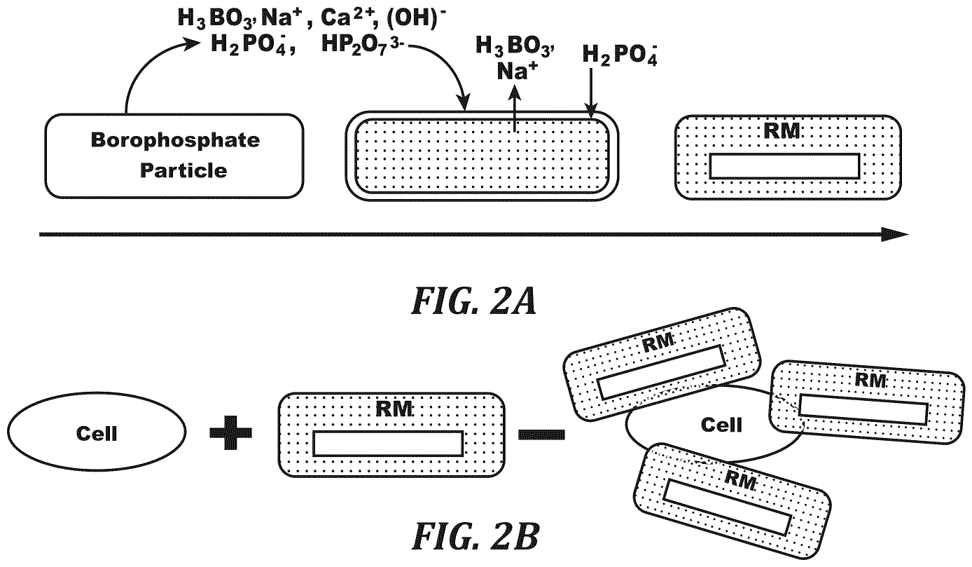

FIGS. 2A and 2B schematically illustrate an example method of making a composition according to embodiments of the present disclosure. In this regard, FIG. 2A is a diagram of particle degradation, ion release, and reaction matrix (RM) formation. Left, borophosphate particle reacts in PSS by releasing ionic species. Middle, released ions react with SBF to form a biocompatible RM of specialized calcium phosphate on the particle surface. Right, the particle is fully reacted to form a hollow precipitate composed of RM and pyrophosphate anions; the PA in the RM inhibit the formation of hydroxyapatite leaving the biocompatible RM. As shown in FIG. 2B, RM particles bind to adhesion proteins upon cells preventing or limiting them from sticking to each other and promoting implantation within myofascial compartments.

In an embodiment, the mixture further comprises about 2 wt. % to about 10 wt. % CoO, about 0.5 wt. % to about 2 wt. % ZnO, about 0.1 wt. % to about 1 wt. % CuO, or a combination thereof.

In an embodiment, the method further comprises calcining the mixture to evolve water prior to the melting.

In an embodiment, the mixture further comprises phosphoric acid.

In an embodiment, melting the mixture is at a temperature in a range of about 900° C. to about 1150° C.

In an embodiment, the method further comprises grinding the bioactive glass composition to provide particles of the bioactive glass composition.

In an embodiment, the particles comprise a largest size of less than about 10 micrometers, less than about 9 micrometers, less than about 8 micrometers, less than about 7 micrometers, less than about 6 micrometers, less than about 5 micrometers, less than about 4 micrometers, less than about 3 micrometers, less than about 2 micrometers, less than about 1 micrometers, or smaller.

In an embodiment, combining the cells and the bioactive glass composition comprises suspending the particles of the bioactive glass composition and the cells in a liquid, such as a liquid configured for or otherwise suitable for injection into a subject.

In an embodiment, the liquid is a sterile saline solution.

In an embodiment, the liquid comprises sodium alginate.

In an aspect, the present disclosure provides a composition made according to the methods of any embodiment of the present disclosure.

In another aspect, the present disclosure provides a composition comprising cells; and a bioactive glass composition comprising an amorphous calcium polyphosphate phase.

In an embodiment, the cells comprise stem cells and the stem cells comprise immortalized stem cells. In an embodiment, the stem cells comprise myoblasts. In an embodiment, the stem cells comprise myogenic stem cells. In an embodiment, the stem cells comprise dystrophin-producing stem cells.

In an embodiment, the amorphous calcium polyphosphate phase comprises orthophosphate and pyrophosphate anions.

In an embodiment, the bioactive glass composition is made according to the methods of any embodiments of the present disclosure.

In another aspect, the present disclosure provides a suspension comprising a liquid; and a composition according to any embodiment of the present disclosure.

In an embodiment, the liquid is a sterile saline solution. While sterile saline solutions are described, it will be understood that other liquids, such as other liquids suitable for injection, are possible and within the scope of the present disclosure. In an embodiment, the liquid further comprising sodium alginate.

In yet another aspect, the present disclosure provides a method of treating injured or diseased tissue, the method comprising contacting the injured or diseased tissue with a composition of any embodiment of the present disclosure or a suspension of any embodiment of the present disclosure. The diseased or injured tissue can comprise various types of tissue disposed in various portions of a subject, such as tissue treatable by injected stem cells. In an embodiment and as shown further herein, the tissue can comprise muscle tissue. In other embodiments and as also shown further herein, the tissue can comprise articular joint cartilage.

While methods of treatment are described, the present disclosure also includes methods in which the compositions of the present disclosure are injected into tissue, such as to distribute cells in the tissue.

In an embodiment, the composition prevents or limits pocketing and promotes dispersion in injected muscle relative to injection of the cells without the bioactive glass composition, such as measured by cell pocket size.

In an embodiment, any pockets in the muscle comprise a thickness or other dimension of less than 80 micrometers from 1 day to 1 month after injection.

### EXAMPLES

### EXAMPLE 1: SCRM IN MUSCLE TISSUE

The present Example describes making compositions comprising stem cells and reacted matrices and the distribution of such compositions in muscle tissue.

### Methods

Biomaterial Fabrication: Time-Release Ion Matrix (TRIM) was generated by mixing the dry, powdered components in a platinum crucible. Phosphoric acid was added to the dry components, and the material was calcined overnight to evolve water prior to melting at 1000-1150° C. for 60 minutes, stirring with a platinum rod. The TRIM melt was ground to form particles smaller than 10 microns in size. Material composition was verified with mass spectrometry. The TRIM composition used to collect preliminary data was 34.7% B₂O₃, 35.3% P₂O₅, 14.0% CaO, 12.3% and Na₂O. 200 mg of TRIM was incubated at 37° C. in 10 mL of cell culture medium (Dulbecco's Modified Eagle Medium (DMEM) or alpha-MEM [no nucleosides], ThermoFisher, Waltham, MA, USA) under severe agitation for 48 hours, TRIM particles degraded and generated more biocompatible orthophosphate and pyrophosphate particles than what is typically seen in fabricated calcium phosphates. Under these conditions, only the RM remains as confirmed with mass spectrometry, permitting combination with stem cells prior to delivery. To isolate and purify the RM, the solution containing RM was transferred to a 15 mL Falcon Tube for gentle centrifugation for 5 min at 10,000 G. After drawing off the used media, RM is resuspended in sterile saline and vortexed prior to centrifugation for 5 min at 10,000 G. Following removal of saline wash, particles are ready to be suspended with an active cell solution.

Stem cell harvest and delivery: Cryogenically frozen MyoK9 stem cells were thawed, plated, grown to confluency, trypsinized, and purified as described previously. Following gentle spinning, a slurry of RM and cell culture media resuspended the stem cells. Following gentle mixing, the slurry was centrifuged to allow cells and RM to settle together. After 30 min to allow RM and stem cell adherence, RM+Myok9 were re-suspended in sterile saline with 0.05% sodium alginate to be injected into muscles. For mice, ~1,000,000 cells were delivered in each intramuscular injection. For pigs, ~33,000,000 cells were delivered intramuscularly.

Biomaterial delivery: For mice, RM (250 μg) was combined with stem cells and suspended in 50 μL sterile saline with 0.05% sodium alginate and injected into the tibial anterior (TA) muscle of anesthetized, immunodeficient mice. Animals were returned to their cage and provided food and water ad libitum. For Becker muscular dystrophy pigs, RM (200 mg) was combined with stem cells and suspended in 10 mL.

Muscle Injury: Muscle injury induces a proper niche for stem cell transplantation. A mouse was anesthetized with isoflurane via a nebulizer and placed on a warming plate to maintain body temperature (37° C.). Hair was shaved over the TA. Muscle injury was induced by injecting 50 μL of 1.2% BaCl₂ through a 30-gauge needle positioned in the left TA. The mouse was kept warm and monitored throughout recovery until fully awoken, then returned to its cage.

For muscle injury in the Becker muscular dystrophy pig, we performed a routine biopsy procedure of the right pelvic limb to remove a discrete amount of muscle prior to introducing stem cells. The pig was maintained on inhaled isoflurane under veterinary care and monitored during the procedure. A two-inch-long incision was made overlying the biceps femoris muscle. Following biopsy of a 1 cm by 2 cm section of muscle, the muscle was sutured with 2-0 Vicryl, followed by myok 9 stem cell injection into the biopsied and non-biopsied (immediately surrounding biopsy site) muscle tissue. The animal was awoken and following ambulation, the pig was placed back in their home cage under daily, routine monitoring.

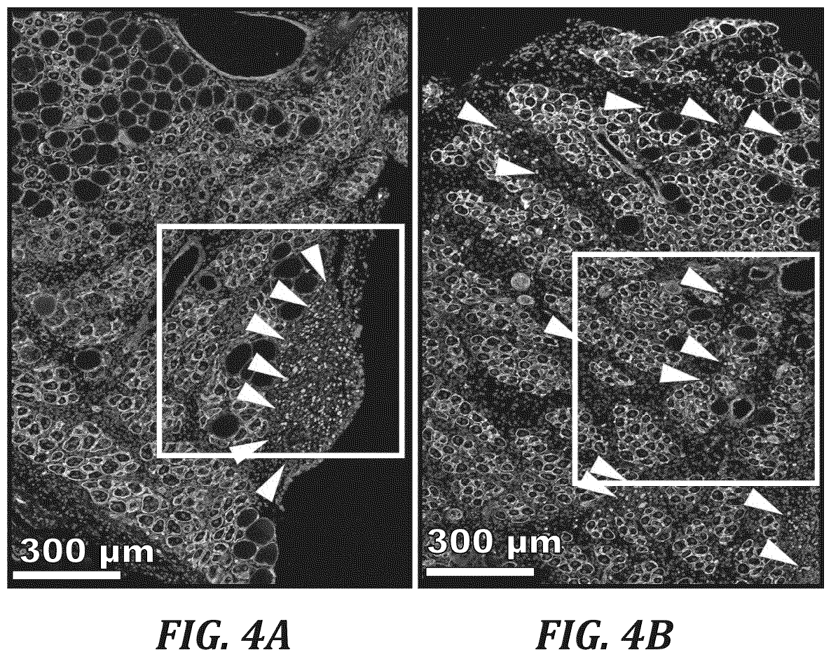

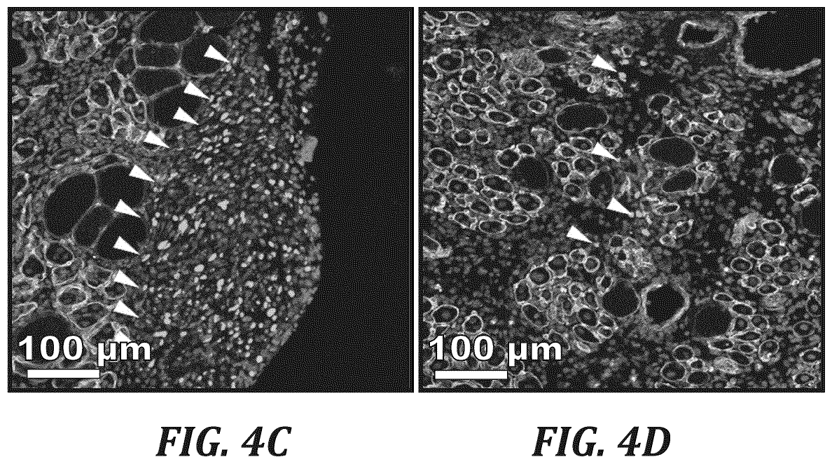

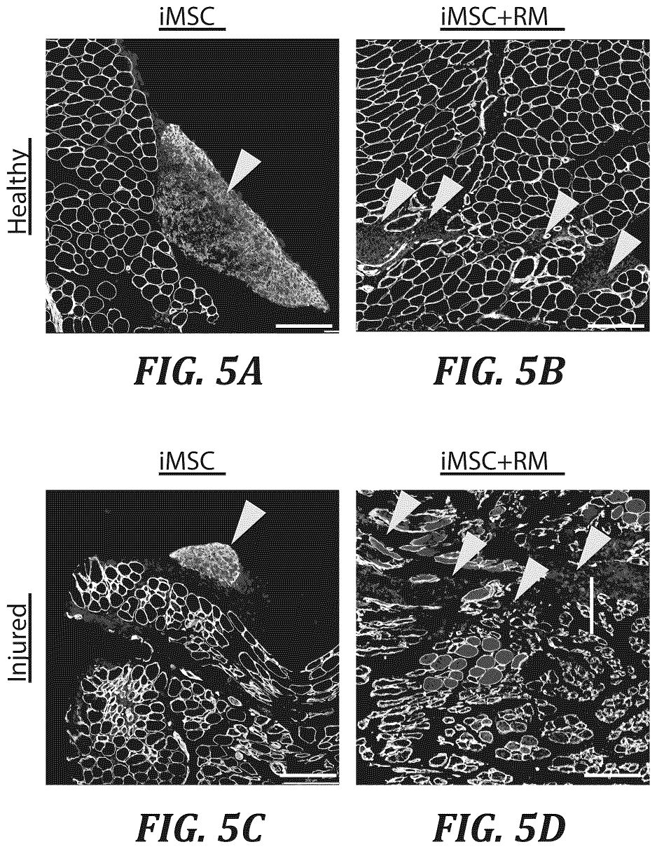

Experimental Animals: Adult 3-month-old, n=2, NOD. Cg-PrkdcSCID (SCID) were used in these experiments as the SCID mouse is immunodeficient and readily accepts MyoK9 (FIGS. 4B and 4D) stem cell or human iMSC (FIG. 5B) without rejection. The left legs of mice were injured (as indicated above) 3 days prior to injection while the right was used as control. One mouse was selected to receive stem cells alone while an additional mouse received the stem cell+RM (SCRM) injection. Following 24 hours, muscles were harvested and prepared for histological measures as described. To determine the effectiveness of RM-mediated stem cell dispersion in muscular dystrophy pig muscle, baseline biopsy was performed on the biceps femoris muscle of the right pelvic limb. Following baseline biopsy, the biopsy site (marked with tattoo dye) of 15 m.o. old dystrophic pigs was injected with SCRM. Following injection, there was a delay of approximately 1 month for implantation and incorporation prior to biopsy and tissue preparation for histology.

Immunohistochemistry Analysis: Primary antibodies used were mouse anti-SV40 (1:200, ab16879, abcam, Waltham, MA, USA) and rabbit anti-laminin (1:400, Cat. #NC1732938, Fisher Scientific; Hampton, NJ, USA). Secondary antibodies were all from Fisher Scientific (Hampton, NJ, USA): Alexa Fluor 488 Goat anti-mouse (1:133, RRID: AB_2633275, Cat. #PIA32723), Goat anti-rabbit Rhodamine (TRITC) (1:400, RRID: AB_90296, Cat. #AP132RMI).

Mouse TA muscles were blotted dry and embedded in Tissue-Plus™ O.C.T. Compound (Scigen, Fischer Scientific, Hampton, NJ, USA), while pig muscles were simply biopsied prior to freezing in liquid nitrogen-cooled 2-methylbutane (Thermo Fisher Scientific, Waltham, MA, USA) and sectioned (thickness, 10 μm) in a Cryostar NX50 Cryostat (Epredia, Kalamazoo, MI, USA) at −17° C. onto a microscope slide. Sections were fixed with ice-cold 4% paraformaldehyde for 20 minutes, washed 3× in Tris-buffered saline (TBS), then permeabilized in 0.5% Triton X-100. Primary antibodies were incubated for 60 minutes at room temperature (RT) in blocking buffer (Pierce™ Protein-Free Blocking Buffer, Thermo Fisher Scientific; Waltham, MA, USA). Sections were washed 3× in TBS, incubated with secondary antibodies in blocking buffer for 60 minutes at RT, washed 3× in TBS, and mounted in Invitrogen™ ProLong™ Gold antifade reagent with DAPI (Cat. #P36941, Fisher Scientific, Hampton, NJ, USA). Slides were imaged on a Stellaris 5 White Light Laser confocal microscope (Leica Microsystems, Deer Park, IL, USA) using Leica LAS_X software (RRID: SCR_013673, Leica Microsystems, Deer Park, IL, USA).

### Results

### Pre-Reaction Promotes Conversion of Ion Matrix to RM and Binds Stem Cells

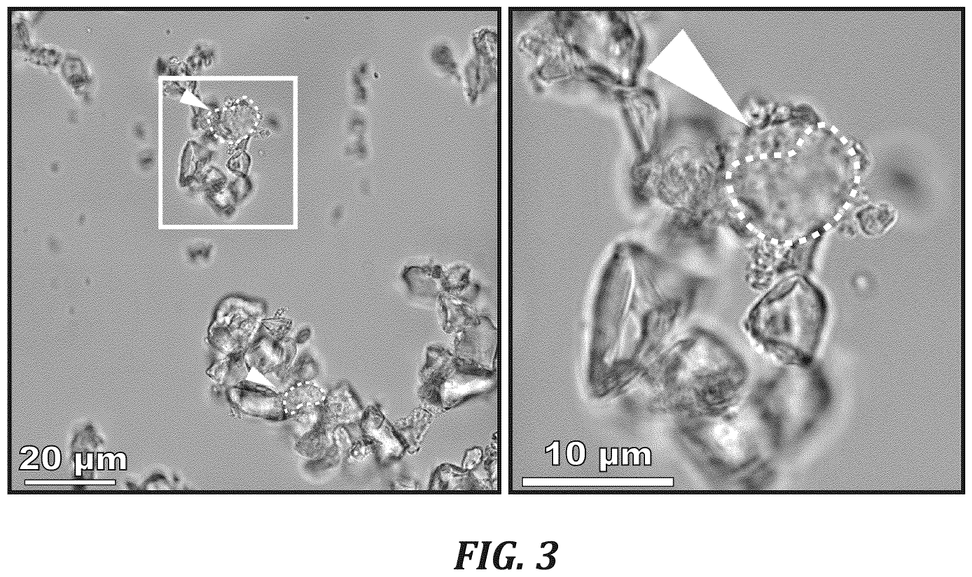

FIGS. 1A and 1B summarize weight dissolution from borophosphate particles, converting to RM. Indeed, upon placement in simulated body fluid or cell culture medium, borophosphate particles began to rapidly react to form the surface reaction comprised of an amorphous calcium polyphosphate (ACpP) phase that includes orthophosphate and pyrophosphate anions (FIGS. 2A and 2B). Upon mixing the RM particles with stem cells, RM particles were seen binding to stem cells, preventing stem cell clumping (FIG. 3).

### Combining Stem Cells with RM Promotes Dispersion in Mice

At 4 days post injury with 1.2% BaCl₂ and 24 hours post injection with either SCRM or stem cells alone, SCID mouse TA muscles demonstrated markedly different histological localization of stem cells (FIGS. 4A-4D). While stem cell injection alone formed a pocket (exceeding 250 μm in thickness, the diffusion distance of oxygen) (see FIGS. 4A and 4C), SCRM injected muscle demonstrated significant dispersion with pockets no greater than 80 μm in thickness, permitting cell survivability and distribution (see FIGS. 4B and 4D).

Combining human stem cells with RM promotes dispersion in mice with and without injury

At 4 post injury with 1.2% BaCl₂ and 24 hours post injection with either iMSC+RM or iMSC alone, SCID mouse TA muscles demonstrated markedly different histological localization of stem cells (FIGS. 5A-5D). While stem cell injection alone formed a pocket outside of the muscle (see FIGS. 5A and 5C), iMSC+RM injected muscle demonstrated significant dispersion within the muscle compartment, permitting cell survivability and distribution (see FIGS. 5B and 5D).

Combining stem cells with RM promotes dispersion in pigs for up to 1 month

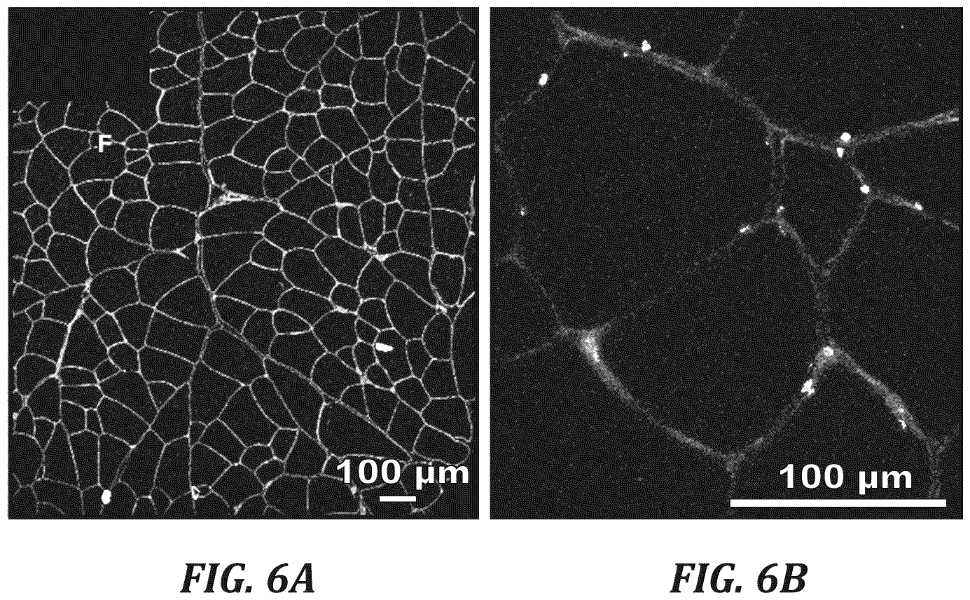

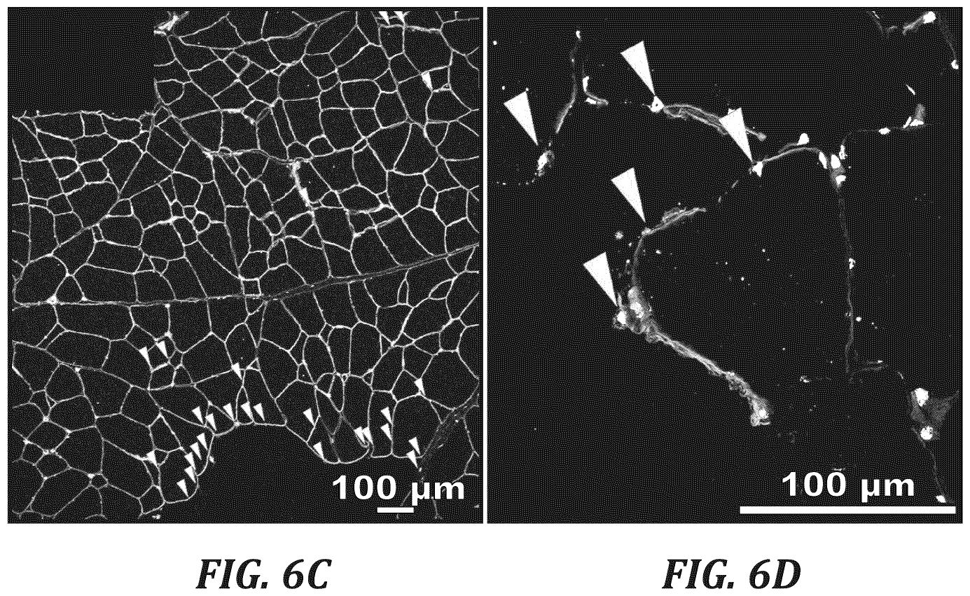

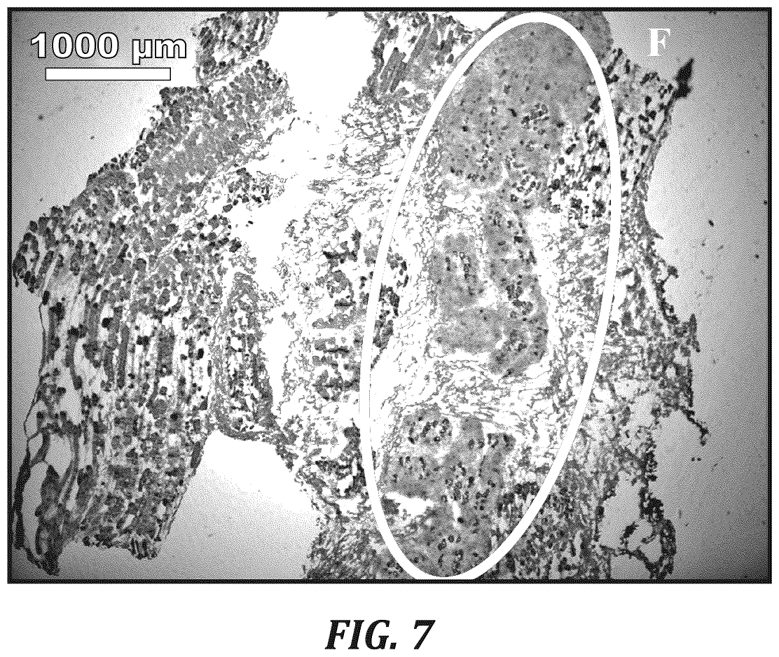

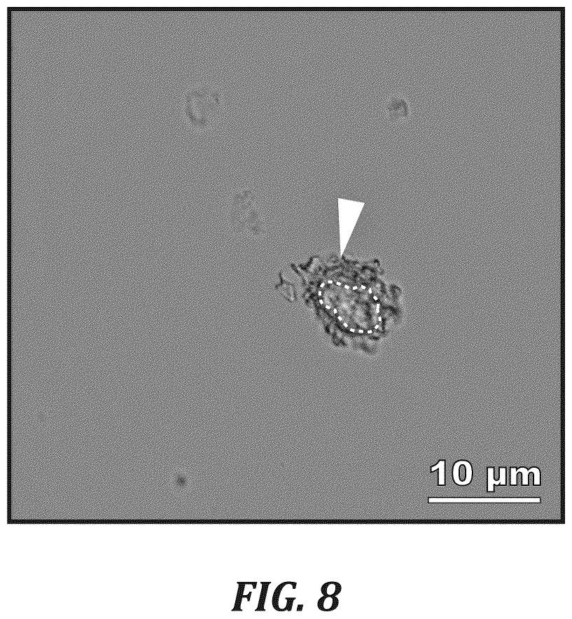

At 1 month post SCRM injection of a biopsy site in Becker muscular dystrophy pig muscle, stem cells were dispersed and demonstrated implantation and survival (FIGS. 6A-6D). That is, myofibers appeared well formed with peripheral myonuclei. The immortalized marker SV-40 which is specific for the stem cells, localized to the nucleus and was clearly visible on multiple fibers (FIGS. 6A-6D). Prior experiments with stem cells alone (no RM) in the canine DMD model demonstrated cell pocketing and death in as little as one week (FIG. 7)

### Discussion

The present disclosure shows widespread stem cell survivability and implantation in both a large and small animal for up to a month post injection. This survivability is due to the combination of stem cells with the bioactive glass compositions, according to embodiments of the present disclosure, to create SCRM prior to injection, thus promoting cell dispersion.

With SCRM, the present disclosure provides a significant breakthrough in combating major roadblocks in cell therapy. The data provided herein strikingly shows dispersion and implantation of intramuscularly injected stem cells into immunodeficient mice and Becker muscular dystrophy pigs. The RM prevents the pocketing (and acute death) and perhaps even provides a niche for implantation of stem cells.

### EXAMPLE 2: REACTED MATRIX COMBINED WITH INDUCED MESENCHYMAL STEM CELLS

The present Example described reacted matrices combined with induced mesenchymal stem cells (iMSCs) and their distribution in articular joint cartilage.

Human iMSCs were originally induced from pluripotent stem cells. These cells were incubated with the iMSCs with RM (made as described in Example 1). It was observed that RM similarly adhered to the outer membrane of iMSCs (FIGS. 5A-5D and 7).

Impactful cellular chaperones can chaperone stem cells in a variety of tissues/organs. Osteoarthritis affects millions and perhaps billions of people worldwide with no effective treatment other than pain management. The present Example demonstrates that RM combined with stem cells according to embodiments of the present disclosure can disperse such stem cells into articular joint cartilage.

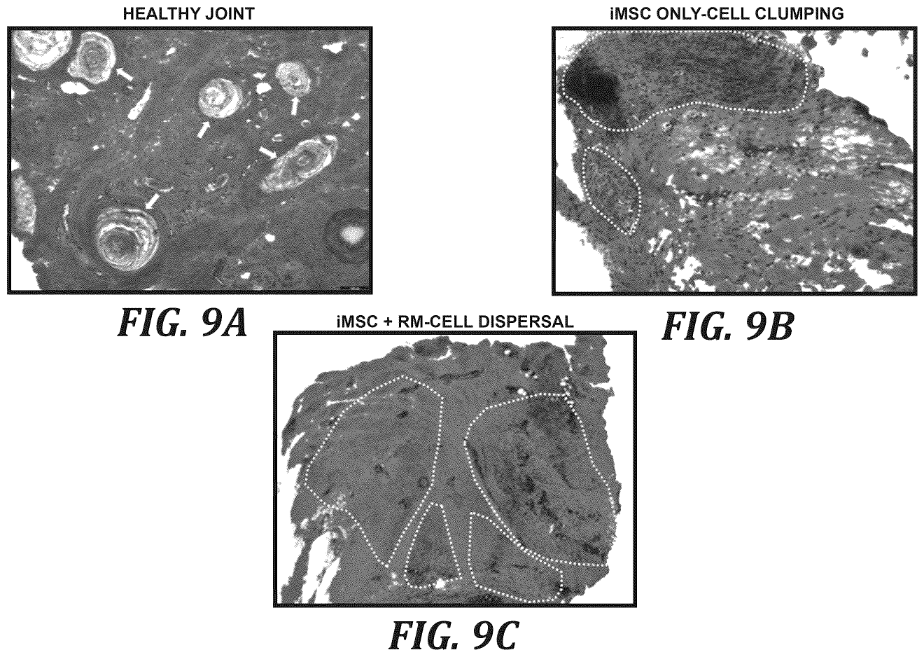

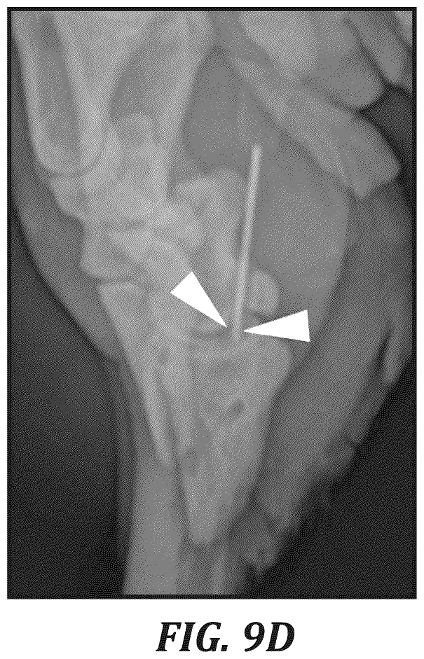

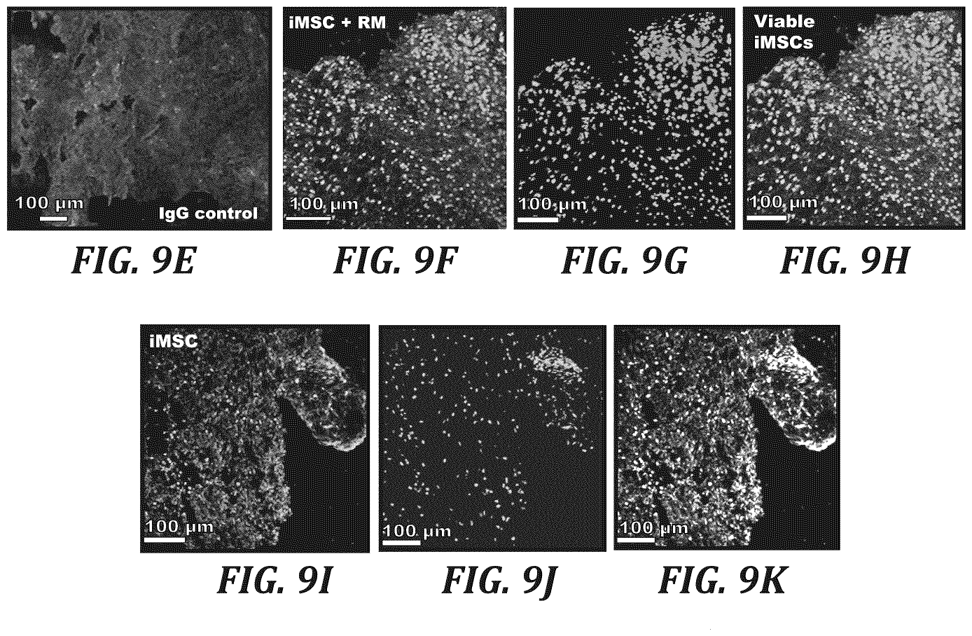

Digital joints of BMD pigs were damaged with BaCl₂ and collagenase type I+II to simulate acutely damaged articular cartilage and provide a stem cell niche. Three days after damage, human iMSCs (~7 million cells/joint)+RM or iMSCs alone were injected into the damaged joints (See FIGS. 9A-9K). Articular injections were guided and confirmed with radiography (FIG. 9D). Healthy, undamaged joints showed rows of healthy chondrocytes (FIG. 9A) while damaged joints revealed dysregulated connective tissue and no noticeable chondrocytes (FIGS. 9B and 9C). Human iMSCs+RM were viable (most localized with nuclei producing a merged color) and super-dispersed into damage articular joint tissue of pigs (FIGS. 9C and 9F-9H) while iMSCs injected alone (without any RM) into a damaged joint appeared clumped in many areas (FIG. 9B) and were less viable (did not localize with nuclei nearly as much) and were not as dispersed as the former (FIGS. 9I-9K).

### EXAMPLE 3: RM COMBINED WITH CANCER CELLS

The present Example describes RM combined with cancer cells and demonstrates that RMs adhere to cancer cells.

### Methods

RM was made as in Example 1.

Cancer cells harvest and delivery: Cryogenically frozen colon cancer cells (colon cancer CT26 adenocarcinoma cells and breast cancer cells MDA-MB-231) were thawed, plated, grown to confluency, trypsinized, and purified as described previously. Following gentle spinning, a slurry of RM and cell culture media resuspended either colon cancer or breast cancer cells. Following gentle mixing, the slurry was centrifuged to allow cells and RM to settle together. After 30 min to allow RM and cancer cell adherence, RM+colon cancer cells or breast cancer cells were re-suspended in sterile saline with 0.05% sodium alginate and Hoechst Dye (34580 Invitrogen) to label cell nuclei to be placed upon a slide for imaging.

### Results

Upon mixing the RM particles with stem cells, RM particles were seen binding to stem cells, preventing stem cell clumping. See FIGS. 10A and 10B.

### Discussion

Cancer cell binding suggests that RM binds broadly to multiple cell types to prevent clumping.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, approximating language may be applied to modify any quantitative representation that may vary without resulting in a change in the basic function to which it is related. As used herein, the terms “substantially, ”about“, and ”at or about“ mean that the amount or value in question can be the value designated some other value approximately or about the same. It is generally understood, as used herein, that it is the nominal value indicated ±10% variation unless otherwise indicated or inferred. For example, ”about 10%“ may indicate a range of 9% to 11%, and ”about 1″ may mean from 0.9-1.1. Other meanings of “about” may be apparent from the context, such as rounding off, so, for example “about 1” may also mean from 0.5 to 1.4. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise. For example, the expression “from about 2 to about 4” also discloses the range “from 2 to 4.”

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value; they are sufficiently imprecise to include values approximating these ranges and/or values. All ranges are combinable.

The above description of illustrated embodiments of the disclosure, including what is described in the Abstract, is not intended to be exhaustive or to limit the disclosure to the precise forms disclosed. While specific embodiments of, and examples for, the disclosure are described herein for illustrative purposes, various modifications are possible within the scope of the disclosure, as those skilled in the relevant art will recognize.

These modifications can be made to the disclosure in light of the above detailed description. The terms used in the following claims should not be construed to limit the disclosure to the specific embodiments disclosed in the specification. Rather, the scope of the disclosure is to be determined entirely by the following claims, which are to be construed in accordance with established doctrines of claim interpretation.

### NON-LIMITING EMBODIMENTS

While general features of the disclosure are described and shown and particular features of the disclosure are set forth in the claims, the following non-limiting embodiments relate to features, and combinations of features, that are explicitly envisioned as being part of the disclosure. The following non-limiting embodiments contain elements that are modular and can be combined with each other in any number, order, or combination to form a new non-limiting embodiment, which can itself be further combined with other non-limiting embodiments.

1. A method of making a composition, the method comprising:
combining cells and a bioactive glass composition comprising an amorphous calcium polyphosphate phase.

2. The method of Embodiment 1, further comprising:
preparing a mixture comprising:
about 10 wt. % to about 40 wt. % B₂O₃;
about 15 wt. % to about 40 wt. % P₂O₅;
about 10 wt. % to about 25 wt. % CaO; and
about 5 wt. % to about 20 wt₂ % Na₂O; and
melting the mixture at a temperature and for a time sufficient to provide the bioactive glass composition.

3. The method of Embodiment 2, wherein the mixture further comprises about 2 wt. % to about 10 wt. % CoO, about 0.5 wt. % to about 2 wt. % ZnO, about 0.1 wt. % to about 1 wt. % CuO, or a combination thereof.

4. The method of Embodiment 2, further comprising calcining the mixture to evolve water prior to the melting.

5. The method of any of Embodiments 1-4, wherein the mixture further comprises phosphoric acid.

6. The method of any of Embodiments 1-5, wherein melting the mixture is at a temperature in a range of about 900° C. to about 1150° C.

7. The method of any of Embodiments 1-6, further comprising grinding the bioactive glass composition to provide particles of the bioactive glass composition.

8. The method of Embodiment 7, wherein the particles comprise a largest size of less than about 10 micrometers.

9. The method of Embodiment 7, wherein combining the cells and the bioactive glass composition comprises suspending the particles of the bioactive glass composition and the cells in a liquid.

10. The method of Embodiment 9, wherein the liquid is a sterile saline solution.

11. The method of Embodiment 9, wherein the liquid comprises sodium alginate.

12. A composition made according to the method of any of Embodiments 1-11.

13. A composition comprising:
cells; and
a bioactive glass composition comprising an amorphous calcium polyphosphate phase.

14. The composition of Embodiment 13, wherein the bioactive glass composition comprises particles of the bioactive glass composition, wherein the particles comprise a largest size of less than about 10 micrometers.

15. The composition of any of Embodiments 13-14, the cells comprise stem cells, and wherein the stem cells comprise immortalized stem cells.

16. The composition of Embodiment 15, wherein the stem cells comprise myoblasts.

17. The composition of Embodiment 15, wherein the stem cells comprise myogenic stem cells.

18. The composition of Embodiment 15, wherein the stem cells comprise dystrophin-producing stem cells.

19. The composition of any of Embodiments 13-18, wherein the cells comprise cancer cells.

20. The composition of any of Embodiments 13-19, wherein the amorphous calcium polyphosphate phase comprises orthophosphate and pyrophosphate anions.

21. The composition of any of Embodiments 13-20, wherein the bioactive glass composition is made according to the methods of any of Embodiments 1-11.

22. A suspension comprising:
a liquid; and
the composition according to any of Embodiments 13-21.

23. The suspension of Embodiment 22, wherein the liquid is a sterile saline solution.

24. The suspension of any of Embodiments 22 or 23, further comprising sodium alginate.

25. A method of treating injured or diseased muscle, the method comprising:
contacting the injured or diseased muscle with the composition of any of Embodiments 12-21 or the suspension of any of Embodiments 22-24.

26. The method of Embodiment 25, wherein the composition prevents or limits pocketing, and promotes dispersion in injected tissue relative to injection of the cells without the bioactive glass composition.

27. The method of any of Embodiments 25 or 26, wherein any pockets in the tissue comprise a thickness of less than 80 micrometers 1 month after injection.

28. The method of any of Embodiments 25-27, wherein the tissue comprises muscle.

29. The methods of any of Embodiments 25-28, wherein the tissue comprises articular joint cartilage. While illustrative embodiments have been illustrated and described, it will be appreciated that various changes can be made therein without departing from the spirit and scope of the invention.

The embodiments of the invention in which an exclusive property or privilege is claimed are defined as follows:

1. A method of making a composition, the method comprising:
combining cells and a bioactive glass composition comprising an amorphous calcium polyphosphate phase.

2. The method of claim 1, further comprising:
preparing a mixture comprising:
about 10 wt. % to about 40 wt. % B₂O₃;
about 15 wt. % to about 40 wt. % P₂O₅;
about 10 wt. % to about 25 wt. % CaO; and
about 5 wt. % to about 20 wt₂ % Na₂O; and
melting the mixture at a temperature and for a time sufficient to provide the bioactive glass composition.

3. The method of claim 2, wherein the mixture further comprises about 2 wt. % to about 10 wt. % CoO, about 0.5 wt. % to about 2 wt. % ZnO, about 0.1 wt. % to about 1 wt. % CuO, or a combination thereof.
The method of claim 2, further comprising calcining the mixture to evolve water prior to the melting.

5. The method of claim 1, wherein the mixture further comprises phosphoric acid.

6. The method of claim 1, wherein melting the mixture is at a temperature in a range of about 900° C. to about 1150° C.

7. The method of claim 1, further comprising grinding the bioactive glass composition to provide particles of the bioactive glass composition.

8. The method of claim 7, wherein the particles comprise a largest size of less than about 10 micrometers.

9. The method of claim 7, wherein combining the cells and the bioactive glass composition comprises suspending the particles of the bioactive glass composition and the cells in a liquid.

10. The method of claim 9, wherein the liquid is a sterile saline solution.

11. The method of claim 9, wherein the liquid comprises sodium alginate.

12. A composition made according to the method of claim 1.

13. A composition comprising:
cells; and
a bioactive glass composition comprising an amorphous calcium polyphosphate phase.

14. The composition of claim 13, wherein the bioactive glass composition comprises particles of the bioactive glass composition, wherein the particles comprise a largest size of less than about 10 micrometers.

15. The composition of claim 13, the cells comprise stem cells, and wherein the stem cells comprise immortalized stem cells.

16. The composition of claim 15, wherein the stem cells comprise myoblasts.

17. The composition of claim 15, wherein the stem cells comprise myogenic stem cells.

18. The composition of claim 15, wherein the stem cells comprise dystrophin-producing stem cells.

19. The composition of claim 13, wherein the cells comprise cancer cells.

20. The composition of claim 13, wherein the amorphous calcium polyphosphate phase comprises orthophosphate and pyrophosphate anions.

21. The composition of claim 13, wherein the bioactive glass composition is made according to the method of claim 1.

22. A suspension comprising:
a liquid; and
the composition according to claim 13.

23. The suspension of claim 22, wherein the liquid is a sterile saline solution.

24. The suspension of claim 22, further comprising sodium alginate.

25. A method of treating injured or diseased muscle, the method comprising:
contacting the injured or diseased muscle with the composition of claim 13 or the suspension of claim 22.

26. The method of claim 25, wherein the composition prevents or limits pocketing, and promotes dispersion in injected tissue relative to injection of the cells without the bioactive glass composition.

27. The method of claim 25, wherein any pockets in the tissue comprise a thickness of less than 80 micrometers 1 month after injection.

28. The method of claim 25, wherein the tissue comprises muscle.

29. The methods of claim 25, wherein the tissue comprises articular joint cartilage.
